(19) United States
(12) Patent Application Publication
(10) Pub. No.: US 2007/0092903 A1
(43) Pub. Date: Apr. 26, 2007

(54) One-color microarray analysis methods, reagents and kits

(76) Inventors: Stephanie B. Fulmer-Smentek, Cupertino, CA (US); Anne Bergstrom Lucas, Mountain View, CA (US); Erik L. Bjeldanes, Danville, CA (US); Petula N. D'Andrade, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,581

(22) Filed: Oct. 13, 2006

Related U.S. Application Data
(60) Provisional application No. 60/729,963, filed on Oct. 24, 2005.

(51) Int. Cl. C12Q 1/68; C12M 3/00

(52) U.S. Cl. 435/6; 435/287.2

(57) ABSTRACT

Microarray platforms for performing one-color and two color analyses using a single platform are provided. Methods of using the microarray platforms and analyzing data obtained from such microarrays are also provided.

### CROSS-REFERENCE TO RELATED APPLICATIONS

This is a nonprovisional application based on U.S. Provisional Application No. 60/729,963, filed Oct. 24, 2005, the disclosure of which is incorporated herein by reference in its entirety.

### BACKGROUND

Gene expression or genomic (e.g., comparative genomic hybridization) analysis can be performed by one-color (intensity-based) or two-color (ratio-based) microarray platforms depending on the specific applications. A one-color procedure involves the hybridization of a single sample to each microarray after if has been labeled with a single fluorophore, whereas in a two-color experiment, two samples (experimental and control) are labeled with different fluorophores and hybridized to a single microarray.

The two-color approach was developed to reduce errors associated with microarray manufacture, but this is less of a problem with the availability of high quality commercial microarrays with decreased variability. The principal advantage of the two-color system is that it allows direct comparison between two samples on a microarray, minimizing errors due to experimental microarray processing. However, dye-specific biases can significantly impact results when using the two-color approach. Although these biases can be mitigated via dye-swapping, or fluorophore reversals, this requires technical replication that adds to the experimental costs of a microarray analysis.

A one-color approach, when paired with high quality microarrays and a robust workflow, offers more flexibility in experimental design with the possibility of reduced error in complex comparisons. Hybridization of a single sample per microarray (rather than two samples) facilitates comparisons across microarrays and between groups of samples. Data variability across assays due to multiple sources of variability, including microarray fabrication and processing, can be reduced for one-color methods, by performing sufficient biological and technical replicate assays. Two of the major requirements of a microarray platform are: the ability to conduct high confidence experiments with reliable system reproducibility, and the ability to detect significant gene expression changes through high sensitivity.

### SUMMARY

The present disclosure describes microarray platforms and/or protocols capable of use in multiple detection modes. In embodiments, a platform can be used in a first detection mode (i.e., a one color mode). In this mode, target nucleic acids in two or more samples are labeled with a single label, and the at least two samples are hybridized to at least two microarrays, wherein each microarray is hybridized with a single sample. Monitoring and/or measuring the signal intensity from probe features on each microarray detects the presence or relative concentration of the target nucleic acids in each sample. In some embodiments, the same single label is used with at least two of or all of the samples. In other embodiments, the same microarray platform can be used in a second detection mode (i.e., a two color mode). In this mode, target nucleic acids in two or more samples are each labeled with at least two different labels, and each sample is hybridized to the same microarray. Measuring the signal intensity from all channels of one microarray detects the presence or relative concentration of target nucleic acids in each sample.

Methods of using the same microarray platform in multiple detection modes are also provided herein. In embodiments, the methods comprise operating the platform in a first detection mode, by labeling target nucleic acids in two or more samples with a single label, wherein the same label is the same label for each sample, followed by hybridization of the labeled samples to two or more microarrays in the platform, wherein each microarray is hybridized to a single sample and measuring and/or monitoring the signal intensity from each probe feature on the microarrays. In other embodiments, the methods comprise operating the platform in a second detection mode, by labeling target nucleic acids in two or more samples with at least two different labels, each label having at least one distinguishable characteristic, hybridizing the labeled nucleic acids to one or more microarrays, wherein each microarray is hybridized with the two or more samples, and monitoring the signal intensity from all probe features in each measurable channel of one or more microarrays in the platform.

### BRIEF DESCRIPTION OF THE FIGURES

FIG. 1 shows intra array % CV of the replicated probes per microarray. The % CV for both the replicated biological probes and the one color spike-in probes are shown. X axis represents the individual microarray experiment, named after the RNA sample hybridized. Three replicate experiments are plotted next to one another. The experiments were done in one color mode.

FIG. 2 shows median inter array % CV. X axis represents the individual microarray experiment named after the RNA sample hybridized. Y axis represents the median % CV of the normalized signals of 3 replicate microarrays. The median % CV of the non-control probes are shown. The experiments were done in one color mode.

### DESCRIPTION

It is to be understood that the compositions and methods described herein are not limited to specific method steps, arrays, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like.

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA.

The term “nucleic acid” refers to a large molecule composed of nucleotide subunits and encompasses both RNA and DNA. A “nucleotide” refers to a subunit of a nucleic acid and has a phosphate group, a 5-carbon sugar and a nitrogen-containing base, as well as functional analogs (whether synthetic or naturally occurring) of such subunits which in the polymer form (as a polynucleotide) can hybridize with naturally occurring polynucleotides in a sequence-specific manner analogous to that of two naturally-occurring polynucleotides. Nucleotide subunits of deoxyribonucleic acids are deoxyribonucleotides, and nucleotide subunits of ribonucleic acids are ribonucleotides.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid references a liquid containing either a biomonomer or biopolymer, respectively (typically in solution).

An “oligonucleotide” generally refers to a nucleotide multimer of about 10 to 100 nucleotides in length, while a “polynucleotide” includes a nucleotide multimer having any number of nucleotides.

An “oligonucleotide probe” refers to a moiety made of an oligonucleotide or polynucleotide, containing a nucleic acid sequence complementary to a nucleic acid sequence present in a portion of a polynucleotide such as another oligonucleotide, or a target nucleic acid sequence, such that the probe will specifically hybridize to the target nucleic acid sequence under appropriate conditions.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand “probe features”, (i.e. an element or spot on an array that is made up of chemical moieties such as nucleic acid sequences, for example) or even more than one hundred thousand features, in an area of less than 20 cm² or even less than 10 cm². The terms “feature” and “probe feature” are used interchangeably herein. For example, probe features may have widths (that is, diameter, for a round spot) in the range of from about 10 μm to about 1.0 cm. In other embodiments each probe feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given probe feature is made up of chemical moieties, e.g., nucleic acids, that bind to (e.g., hybridize to) the same target (e.g., target nucleic acid), such that a given feature corresponds to a particular target. At least some, or all, of the probe features have different sequences than some, or all, of the other probe features on a given array. For example, when any repeats of each feature composition are excluded the remaining features may account for at least 5%, 10%, or 20% of the total number of features. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array.

As used herein, a “target nucleic acid sample” or “target nucleic acids” refer to nucleic acids comprising sequences whose quantity or degree of representation (e.g., copy number) or sequence identity is being assayed. The term can refer to genomic nucleic acids comprising sequences whose quantity or degree of representation (e.g., copy number) or sequence identity is being assayed (as in a CGH analysis, for example), or to DNA or RNA sequences whose relative concentration is being determined, as in a gene expression assay.

An “array platform” or “microarray platform” refers to a base system for performing microarray analysis, comprising one or more microarrays with oligonucleotides immobilized on the array substrate. The platform also comprises all systems and reagents required for hybridization and detection of samples on the microarray and all systems and reagents required for processing, scanning and analysis of the microarrays. A “microarray protocol” refers to the methods and/or steps utilized for processing, scanning, and/or analysis of the microarrays.

The term “detection mode” refers to the method used to detect the presence, abundance or relative concentration of a target nucleic acid in a sample. A detection mode comprises steps for labeling the targets with appropriate reagents that are detectable using standard techniques, and steps for binding or hybridizing the targets to a microarray. The detection mode typically also comprises measuring, observing or monitoring the signal intensity from the microarray in order to detect the presence, abundance or concentration of the targets.

A “detectable characteristic” refers to a property of a label used with a target nucleic acid to be monitored, measured or observed by standard method of detection used for microarray analysis. For example, a reagent used to label a nucleic acid may be a fluorescent tag, with a particular fluorescent color as the detectable characteristic. Where two or more labels are used, the detectable characteristics of the labels are distinguishable from each other. For example, when two fluorescent tags are used to label nucleic acid samples, the two tags will have different fluorescent colors, thereby providing distinguishable detectable characteristics. A “measurable channel” refers to the signal generated by a distinguishable characteristic of a label. For example, a measurable channel could consist of a wavelength at which a given fluorophore emits light.

The term “% CV” refers to coefficient of variation, which is defined as the ratio of the standard deviation to an average value, expressed as a percentage. % CV functions as a metric that can be used to assess the reproducibility or quality of a microarray. A quality control report based on the detection of targets hybridized to a microarray provides assessment of the performance of the array in a particular experiment for criteria including, but not limited to, signal to noise, dynamic range, linearity of response, and background.

“Hybridizing” and “binding”, with respect to polynucleotides, are used herein interchangeably.

A stringent hybridization and stringent hybridization wash conditions in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M followed by washes of 0.5×SSC and 0.1×SSC at room temperature, or by washing with 0.6×SSPE and 0.06×SSPE at elevated temperature for 1 minute.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “predetermined” refers to an element whose identity or composition is known prior to its use. For example, a “predetermined temperature” is a temperature that is specified as a given temperature prior to use. An element may be known by name, sequence, molecular weight, its function, or any other attribute or identifier. As used herein, “automatic”, automatically”, or other like term references a process or series of steps that occurs without further intervention by the user, typically as a result of a triggering event provided or performed by the user.

As used herein, the term “signal” refers to the detectable characteristic of a detectable molecule. Exemplary detectable characteristics include, but are not limited to: a change in the light adsorption characteristics of a reaction solution resulting from enzymatic action of an enzyme attached to a labeling probe acting on a substrate; the color or change in color of a dye; fluorescence; phosphorescence; radioactivity; or any other indicia that can be detected and/or quantified by a detection system being used.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination, which will perform those, or equivalent steps. Programming may be accomplished, for example, from a computer readable medium carrying necessary program code (such as a portable storage medium) or by communication from a remote location (such as through a communication channel).

A “memory” or “memory unit” refers to any device that can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box), which will provide additional protection to items contained in the package from exposure to the external environment. In the case of just a single array assembly a package may be that array assembly with some protective covering over the array assembly (which protective cover may or may not be an additional part of the array unit itself).

Microarray Platforms and Method of Use with Multiple Detection Modes

Microarray platforms and/or protocols capable of use in multiple detection modes are provided herein. In embodiments, one of the detection modes comprises labeling of target nucleic acids in each of two or more samples with a single label with a detectable characteristic. In some embodiments, the same label is used with each sample. The labeled samples are then hybridized to two or more microarrays in the platform, with each microarray hybridized with a single sample. Monitoring and/or measuring the relative signal intensity between different microarrays in the platform provides an indication of the presence, abundance or relative concentration of target nucleic acids in the samples. In other embodiments, the detection mode comprises labeling target nucleic acids in two or more samples with different labels having distinguishable detectable characteristics. The differentially labeled samples are then hybridized to one or more microarrays in the platform, with at least two or all the labeled samples hybridized to the same microarray. Monitoring and/or measuring the relative signal intensity from all the channels on the same microarray provides an indication of the presence, abundance or relative concentration of target nucleic acids in the samples. The same microarray platform may be used in either detection mode.

In some embodiments, the multiple detection modes used with the microarray platform comprise a one color detection mode and a two color detection mode. In an aspect, the multiple detection mode platform is made by modifying or optimizing a two color microarray platform for one color experiments, or by optimizing a one-color platform for two color experiments. For example, but not limiting to the description herein, in a two color platform, samples must be labeled prior to hybridization, to allow the labels to be distinguished from one another, because the samples are hybridized to the same microarray. When the same platform is used in the one color mode, the samples may be directly labeled prior to hybridization of each of the two or more samples to different microarrays, or after hybridization, because the samples are distinguished by which microarray each is hybridized to, rather than by differential labeling.

In embodiments, the same microarray platform may be used in either one color or two color modes. When the same platform is used with the one color mode, each sample of the at least two different samples are each hybridized to a separate microarray. In embodiments, the inter array % CV has a maximum value of about 20% % CV in replicate experiments. In some embodiments, the inter array median % CV of the microarrays in the platform is about 20%, 19%, 18%, 17%, 16%, 15%, 14%, 13%, 12%, 11%, 10%, 9%, 8%, 7%, 6%, 5%, 4%, 3%, 2%, 1% or less. In some embodiments, the intra array % CV is about 15%, 14%, 13%, 12%, 11%, 10%, 9%, 8%, 7%, 6%, 5%, 4%, 3%, 2%, 1% or less.

In some embodiments, where same platform is used with the two color mode of detection, at least two or all of the samples are hybridized on the same microarray, depending on the number of samples analyzed, more than one microarray may be utilized, but at least two of the samples are hybridized to each microarray. In some embodiments, the inter array median % CV of the microarrays in the platform is about 20%, 19%, 18%, 17%, 16%, 15%, 14%, 13%, 12%, 11%, 10%, 9%, 8%, 7%, 6%, 5%, 4%, 3%, 2%, 1% or less. In some embodiments, the intra array % CV is about 15%, 14%, 13%, 12%, 11%, 10%, 9%, 8%, 7%, 6%, 5%, 4%, 3%, 2%, 1% or less.

In embodiment, the microarray, when utilized in the one color modes, utilizes spike in controls as described in application entitled “Spike-in Compositions and Methods”, (Ser. No. not yet assigned), filed evendate herewith, and hereby incorporated by reference. In some embodiments, the one color mode microarray platform has a dynamic range of detection of at least 3-6 orders of magnitude.

In some embodiments, the microarray platform is a gene expression microarray, a CGH array whole genome, CpG island, promoter arrays, snp, or splice array.

The present description also provides methods for using a microarray platform in multiple detection modes. In embodiments, the subject methods comprise operating the platform in either a first or second detection mode (i.e., a one color or two color method). The first detection mode labeling target nucleic acids in two or more samples with a single label having a single detectable characteristic, followed by hybridization of the labeled samples to two or more microarrays in the platform, and monitoring and/or measuring of the signal intensity from each probe feature on the microarrays, wherein each microarray is hybridized with a single sample. The second detection mode comprises labeling target nucleic acids in two or more samples with at least two different distinguishable labels, and measuring intensity of all probe features in each measurable channel of one or more microarrays in the platform, wherein each microarray is hybridized with multiple samples. In aspects, the relative signal intensities between microarrays in the first detection mode indicate the relative concentration of target nucleic acids in samples hybridized to different microarrays. In other aspects, the relative signal intensities within a single microarray in the second detection mode indicate the relative concentration of target nucleic acids in samples hybridized to the same microarrays.

The described methods comprise a step wherein the target nucleic acids are labeled with a label having a detectable characteristic. In embodiments, the target nucleic acids are labeled with fluorescent dyes or fluorophores, such as phycoerythrin, cyanine-3, and cyanine-5, for example. In an aspect, when the first detection mode (i.e. one color) is used, the samples are labeled with just one fluorophore (for example, Cy-3). In another aspect, when the second detection mode (i.e., two color) is used, the samples are simultaneously labeled with two fluorophores which are distinguishable from each other (for example, Cy-3 and Cy-5). In embodiments, the subject methods comprise direct labeling of the nucleic acids, prior to hybridization. In an aspect, the nucleic acids are amplified prior to labeling.

The subject platforms, protocols, and methods are useful for analysis of different types of target nucleic acids. In embodiments, the target nucleic acids analyzed by the subject methods comprise DNA. In aspects, genomic DNA can be analyzed by using the subject platforms and arrays in the second detection mode, or two color mode (such as a CGH analysis, for example). In other embodiments, the target nucleic acids analyzed by the subject methods comprise RNA, including, but not limited to, messenger RNA and micro RNA. In aspects, RNA analysis (i.e. gene expression analysis) can be performed by using the subject platforms and arrays in the first detection mode (or one color mode).

One Color and Two Color Detection

The subject platforms and arrays are capable of use with multiple detection modes. In embodiments, these detection modes comprise either a one color (i.e. intensity-based) or a two color (i.e. ratio-based) mode of detection. A particular detection method is chosen on the basis of the specific application for the microarray (such as the type of nucleic acids to be analyzed, for example). For example, one color methods are often used for gene expression analysis. In the one color mode, each sample containing the target nucleic acids is hybridized to a single microarray. A positive control transcript (or one-color spike in, as described in 60/729,963 (filed Oct. 24, 2005), is added to the sample prior to hybridization, which is optimized to anneal to complementary probes on the microarray, with minimal self-hybridization and cross-hybridization. Hybridization of a single sample per microarray allows comparisons of gene expression data across microarrays and between groups of samples. Intensity-based measurements can also be statistically analyzed (using ANOVA methods, for example), where changes in the intensity across microarrays can be used to determine gene up-regulation or down-regulation. The one color method has the advantage of experimental simplicity and flexibility.

In the two color detection mode, two or more samples containing the target nucleic acids are labeled and hybridized to a single microarray. All data is generated from two samples on the same microarray, which allows for direct comparison between two samples and eliminates errors due to microarray processing. By combining the two detection methods in a single platform, the advantages (in terms of sensitivity) of the two-color methods are added to the ease and flexibility of the one color approach, providing a more powerful and robust method of microarray analysis. A single platform capable of operation in both one color and two color modes has not been previously described.

In embodiments, the two color platform is capable of use in a one color detection mode, because of higher array quality, more consistent microarrays, robust protocols and sufficient quality control procedures. In an aspect, using a one color detection mode on the subject platforms, highly reproducible results, indicating very low variation between arrays, can be obtained, with interarray median % CVs as low as about 10%.

When the same microarray platform is compared within the two color and the one color array replicates with the same samples, the average Pearson correlations for intensity measurements for one color correlation values are about 0.950 or greater, and for two color were about 0.965 or greater. This indicates that using the same platform with different detection methods has a high degree of reproducibility. When the same samples are analyzed by one color and two color assays on the same gene expression platform fold change values correlate well indicating the different detection methods generate similar results with similar levels of sensitivity. Another criteria for evaluation is accuracy. Accuracy can be determined by using an orthogonal gene expression measurement technology, such as TaqMan® (Applied Biosystems) assays, on randomly selected genes mapped in common between the microarray platform and the TaqMan® platform. Assessments of the accuracy of both detection methods indicate a similar level of accuracy. A comparative study of the accuracy and reproducibility of various platforms operated in one color or two color detection modes is provided in Patterson et al., Nature Biotech. 24:1140-1150 (2006), which is incorporated herein by reference.

Methods for Determining Relative Concentration

The present description provides methods for determining the relative concentration of target nucleic acids using multiple detection methods with a single microarray platform. In embodiments, the single microarray platform comprises one or more microarrays. Each microarray includes multiple probe features that comprise oligonucleotides with sequences complementary to the target nucleic acid sequences. The relative concentration of the target nucleic acids is determined as a function of the signal intensity observed from the probe features when samples are labeled and hybridized to the microarrays.

In an embodiment, each sample is labeled with a single label and each sample is hybridized to a separate microarray within the platform. Comparison of the signal intensities from probe features on different microarrays in the platform indicates the relative concentration of nucleic acids in each sample. In some embodiments, for one color experiment, processed signal values are obtained using software such as Feature Extraction Software (Agilent).

In another embodiment, the nucleic acids in each sample are labeled using different labels, each with a single distinguishable characteristic. The labeled samples are then hybridized to one or more microarrays, such that each microarray is hybridized with multiple samples. Comparison of the signal intensities from all of the probe features on a single microarray indicates the relative concentration of nucleic acids in each sample.

For the two-color microarrays, raw data signals are preprocessed in a similar fashion as those for one-color microarray, but included additional preprocessing to adjust for possible dye bias within a microarray.

The methods described herein are used to determine relative concentrations of target nucleic acids in two or more samples. In embodiments, the target nucleic acids in the samples are labeled with reagents that have detectable characteristics. In an aspect, the detectable characteristic is fluorescence, and the reagents used to label the nucleic acids are fluorescent dyes or fluorophores such as, but not limited to, phycoerythrin, cyanine-3 (Cy-3), cyanine-5 (Cy-5), etc. Where a microarray is hybridized with a single sample, the sample is labeled with a single reagent or fluorophore, such as Cy-3, for example. Where each microarray in the platform is hybridized with multiple samples, the sample is labeled with more than one fluorophore, such as Cy-3 and Cy-5, for example. Comparing the fluorescent signal generated by each microarray probe feature with other microarrays in the platform, or within a single microarray, the relative concentration of different nucleic acids between the samples can be determined (because signal intensity and concentration are related). In embodiments, labeling can include use of standard methods for amplification, such as, but not limited to, use DNA primers and polymerases for amplification, for example. The amplification may occur before the nucleic acids have been labeled. In aspects, the amplification may take place concurrently with the labeling step.

In the methods described herein, amplified and labeled target nucleic acids are hybridized to one or more microarrays that comprise the microarray platform. For each microarray, specific volumes of labeled sample are hybridized to the array, using standard reagents and buffers for hybridization, as described above. For example, samples are hybridized to a gene expression microarray at 65° C. for 17 hours. Hybridization is followed by washing with standard wash buffers under standard conditions, as described above. After washing, the microarray is scanned to determine the relative signal intensity in the microarray platform.

The methods described herein can be used for analysis of different types of nucleic acid samples. For example, the detection method where samples are differentially labeled and hybridized to one or more microarrays is useful for analysis of genomic DNA. The detection method where a single sample is hybridized to a microarray is well-suited for analysis of gene expressions due to enhanced flexibility in experimental design as compared to the two color method. Samples used for gene expression analysis can be DNA, RNA, messenger RNA, micro RNA, etc. The methods described herein provide useful techniques for comparing samples across microarrays and between groups of samples. Variability introduced by conducting multiple separate assays is eliminated using the methods described herein.

Arrays

The present description also provides nucleic acid microarrays produced using the subject methods, as described herein. The subject arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another. An exemplary array is described in U.S. Patent Publication No. 20050095596, which is incorporated herein by reference.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

The arrays as described herein can be used in a variety of different microarray applications, including gene expression experiments and genomic analysis. In using an array, the array will typically be exposed to a sample (for example, a fluorescently labeled analyte, such as a sample containing genomic DNA) and the array then read. Reading of the array may be accomplished by illuminating the array and reading the location and intensity of resulting fluorescence at each feature of the array to detect any binding complexes on the surface of the array. For example, a scanner may be used for this purpose that is similar to the AGILENT MICROARRAY SCANNER available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### EXAMPLES

Experimental examples described herein were designed to demonstrate the performance of an inventive one color gene expression protocol developed for use with a microarray platform designed for traditional two color analysis.

Briefly, target nucleic acids in the sample were labeled and hybridized to Agilent Whole Human Genome microarrays. Agilent 60-mer microarrays (Whole Human Genome Microarray kit: P/N G4112A) printed with Agilent SurePrint® technology were used. This microarray has 41,000 human genes and transcripts with one 60 mer oligonucleotide probe representing each sequence. Probes for 75 genes were replicated ten times to allow for intraarray reproducibility measurements.

Two types of human total RNA samples K562 (Ambion, P/N 7832) and MG63, an osteosarcoma cell line (Ambion, P/N 7868), were mixed at the following ratios to generate 11 mixed RNA samples: 100:0; 99:1; 95:5; 90:10; 75:25; 50:50; 25:75; 10:90; 5:95; 1:99; and 0:100. Samples were diluted and amplified according to standard procedures and directly labeled with Cyanine 3-CTP (Perkin Elmer P/N NEL 580). Three replicate labeling reactions were performed for each mixed RNA sample. Samples were hybridized to gene expression microarrays as per standard techniques. Samples were hybridized at 65° C. for 17 hours, and following hybridizations, the microarrays were washed with standard wash buffers (as described herein) at elevated temperature. The microarray was then scanned (using the Agilent scanner, for example) to detect nucleic acid sequences on the basis of signal intensity from the probe features on the microarrays.

Intra-Array Reproducibility

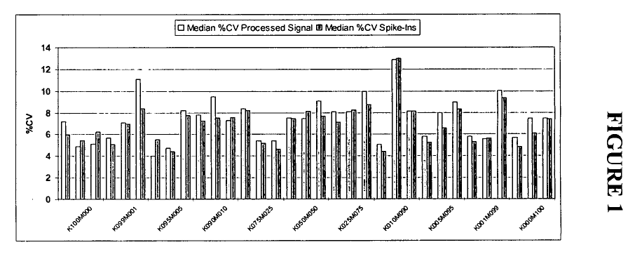

FIG. 1 shows median % CV of the replicated experimental probes per microarray. Intra-array reproducibility (i.e. reproducibility of data from replicated probes on a single array, when the platform developed for two color use is used in the one color mode) is measured by the median % CV of the replicates probes on each microarray. The values of the % CVs shown are reported on the quality control report generated by the Agilent Feature Extraction software. This graph presents the % CV for both the replicated Non-Control probes (light bars) and the One Color Spike-In (i.e. positive controls) probes (dark bars). The average % CV across all 33 microarrays is 7.22%. K000M100 indicates 0% K562+100% MG63.

Inter-Array Reproducibility of Three Replicated Arrays

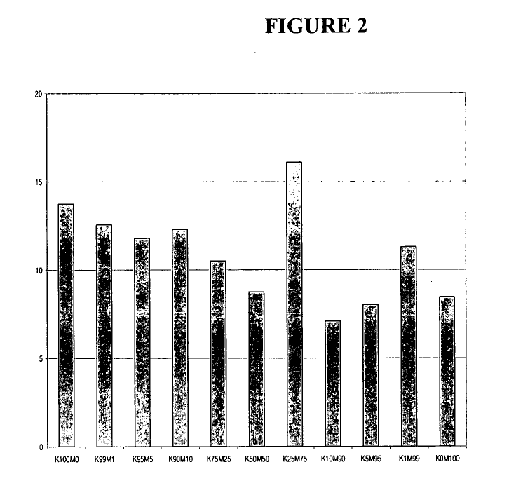

FIG. 2 shows % CV of the normalized signals of the three replicates. Inter-array reproducibility (i.e. the reproducibility of data from samples on different arrays, when the platform developed for two color use is used in the one color mode) is measured by the % CV of the normalized signals at the feature level across the three replicated arrays for each mixed RNA target. The average of the % CV for all of the bars shown on the plot is 10.97%.

Thus, it can be seen from these figures that use of the one color detection mode on the subject platforms (originally developed as two color platforms) produces a % CV of not greater than about 10%. The % CV shown here represents total system variability including labeling, array processing, scanning and data extraction.

With the introduction of the one color mode into a platform designed for two color analysis as described herein, researchers will have unprecedented flexibility in the design of their microarray experiments. As a part of a new multiple detection mode microarray platform, the subject methods provide integrated quality control information for in-process sensitivity and dynamic range determinations, high data confidence from robust system performance, and enhanced sensitivity and experimental flexibility with the capability of running either one color or two color detection modes experiments on the same 60-mer microarray platform.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A microarray platform capable of use in multiple detection modes, the detection modes comprising:
a first detection mode comprising:
labeling of nucleic acids in each of two or more samples with a single label having a single detectable characteristic;
hybridizing the labeled samples to two or more microarrays, wherein each microarray is hybridized with a single sample; and
measuring the signal intensity from each probe feature on the microarrays; or
a second detection mode comprising:
labeling of nucleic acids in each of two or more samples with at least two different labels, each label having at least one distinguishable detectable characteristic;
hybridizing the labeled samples to one or more microarrays, wherein each microarray is hybridized with the two or more samples; and
measuring the signal intensity from each probe feature in all measurable channels on one or more microarrays,
wherein the microarray platform may be used with either detection method for a particular microarray-based experiment.

2. The microarray platform of claim 1, wherein the first detection mode is a one-color detection mode and the second detection mode is a two-color detection mode.

3. The microarray platform of claim 1, wherein a platform developed for operation in the two-color mode is optimized for operation in the one-color mode, or a platform developed for operation in the one-color mode is optimized for operation in the two-color mode.

4. The microarray platform of claim 3, wherein use of the one-color platform in two-color mode comprises labeling the nucleic acids in two or more samples prior to hybridization to the microarrays.

5. The microarray platform of claim 1, wherein the interarray percentage coefficient of variance (% CV) of signal is no greater than about 20% using either detection mode.

6. A method of using a microarray platform in multiple detection modes, the method comprising
operating the platform in a first detection mode, by labeling target nucleic acids in two or more samples with a single label having a single detectable characteristic, by hybridizing the labeled samples to two or more microarrays in the platform, and monitoring the signal intensity from each probe feature on the microarrays, wherein each microarray is hybridized with a single sample; or
operating the platform in a second detection mode, by labeling target nucleic acids in two or more samples with different labels, each label having at least one distinguishable detectable characteristic, hybridizing the labeled samples to one or more microarrays, and a monitoring the signal intensity from all probe features in each measurable channel of one or more microarrays in the platform, wherein each microarray is hybridized with the two or more samples.

7. The method of claim 6, wherein the relative signal intensities between microarrays in the first detection mode indicate the relative concentration of target nucleic acids in samples hybridized to different microarrays.

8. The method of claim 6, wherein the relative signal intensities within a single microarray in the second detection mode indicate the relative concentration of target nucleic acids in samples hybridized to the same microarrays.

9. The method of claim 6, wherein the target nucleic acids in two or more samples are labeled with fluorescent dyes or fluorophores.

10. The method of claim 6, wherein the fluorescent dyes or fluorophores are selected from the group consisting of phycoerythrin, cyanine-3 and cyanine-5.

11. The method of claim 6, wherein the nucleic acids are amplified prior to labeling.

12. The method of claim 6, wherein the target nucleic acids comprise DNA.

13. The method of claim 6, wherein the target nucleic acids comprise genomic DNA.

14. The method of claim 12, wherein the microarray platform is operated in the second detection mode.

15. The method of claim 6, wherein the target nucleic acids comprise RNA.

16. The method of claim 15, wherein the target nucleic acids comprise messenger RNA.

17. The method of 15, wherein the target nucleic acids comprise microRNA.

18. The method of claim 15, wherein the microarray platform is operated in the first detection mode.
